(19) United States
(12) Patent Application Publication
(10) Pub. No.: US 2008/0021209 A1
(43) Pub. Date: Jan. 24, 2008

(54) Ethers of bisanhydrohexitols

(75) Inventors: Anthony East, Madison, NJ (US); Michael Jaffe, Maplewood, NJ (US); Yi Zhang, Harrison, NJ (US); Luiz H. Catalani, Carapicuiba (BR)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(73) Assignee: New Jersey Institute of Technology, Newark, NJ (US)

(21) Appl. No.: 11/809,035

(22) Filed: May 31, 2007

Related U.S. Application Data
(60) Provisional application No. 60/810,060, filed on Jun. 1, 2006.

(51) Int. Cl. C07H 1/00

(52) U.S. Cl. 536/124

(57) ABSTRACT

A novel method for the synthesis of ethers of anhydrosugars, such as isosorbide, isomannide, and isoidide, is disclosed. The bisglycidyl ethers are useful as substitutes for bisphenol A in the manufacture of thermoset epoxy ethers. Anhydrosugar ethers are derived from renewable sources and are not xenoestrogenic. Higher alkyl and aralkyl ethers are stable high-boiling oils that are good plasticizers for materials such as PVC.

### BACKGROUND OF THE INVENTION

The invention relates generally to ethers derived from bisanhydrohexitols, notably isosorbide, isomannide and isoidide, and to an improved method for their synthesis and, more specifically, for their use as plasticizers for materials such as PVC and for polymer intermediates such as the diglycidyl ethers that are intermediates for epoxy thermoset resins.

Polyvinyl chloride (PVC) is a general resin that can attain various physical processing properties by suitably mixing additives such as stabilizers, fillers, pigments, and plasticizers. Polyvinyl chloride with the various physical processing properties is widely used as a material for goods such as wallpaper, gloves, and toys, as well as for pipe, electric wire insulation, and artificial leather. Plasticizers are plastic additives, most commonly phthalates, that give hard plastics like PVC the desired flexibility and durability. Most plasticizers are nonvolatile organic liquids or low-melting point solids, which function by reducing the normal intermolecular forces in a resin thus permitting the macromolecules to slide over one another more freely. They are often based on esters of polycarboxylic acids with linear or branched aliphatic alcohols of moderate chain length. Plasticizers work by embedding themselves between the chains of polymers, space them apart (increasing of the “free volume”), and thus significantly lowering the glass transition temperature for the plastic and making it softer.

Representative plasticizers used in processing of polyvinyl chloride resin include phthalate-based plasticizers, adipate-based plasticizers, and trimellitate-based plasticizers, etc. Phthalic acid diesters(phthalates) are the primary plasticizers for most flexible polymer products, especially polymer products formed from polyvinyl chloride (PVC) and other vinyl polymers. Examples of common phthalate plasticizers include, for example, di-isononyl phthalate (DINP) and di-2-ethylhexyl-phthalate (DEHP). The most commonly used plasticizer, DEHP, is phthalate-based, and it plays a role as a standard plasticizer for performance evaluation of other plasticizers.

Although phthalate plasticizers have been tested for more than 40 years and are among the most studied and best understood compounds in the world from a health and environmental perspective, phthalate plasticizers have recently come under intense scrutiny by public interest groups that are concerned about the potential of adverse health effects in children exposed to these chemicals. Consequently, there is a demand for phthalate-free plasticizers that provide the same properties when added to polymer resins such as, for example, vinyl polymers, rubbers, polyurethanes, and acrylics. Safer plasticizers with better biodegradability and less biochemical effects are being developed. Some such plasticizers include acetylated monoglycerides that are used as food additives and alkyl citrates, used in food packaging, medical products, cosmetics, and children toys.

Bisanhydrohexitol ethers are derived from biomass-derived materials which are known to be biologically compatible and harmless. The ether derivatives described have no ester functions, which could be subject to hydrolysis and degradation during processing or use and hence have obvious advantages as plasticizers.

Methods of making ethers from bisanhydrohexitols, notably isosorbide, have been described. Usually they involve forming the bis alkali metal alkoxide with hazardous and reactive species, such as sodium hydride or sodium metal, and reacting this, without isolation, with alkyl halides or their chemical equivalents, such as sulfonate esters of fatty alcohols. U.S. Pat. No. 3,272,845 (Zech, et al.) describes such a method of making the bisglycidyl ethers of isosorbide, isomannide, and isoidide, which collectively are referred to in the patent as isohexides. Another route involves heating the bisanhydrohexitol with an alkyl carbonate in the presence of a basic catalyst at high temperature under high pressure. An example of this route is described in U.S. Pat. No. 4,770,871 (Greenshields).

The method of the present invention differs from those previously described in the other patents in a number of ways. Notably it uses aqueous caustic alkali at atmospheric pressure in a mixture of specific solvents, as will be described in more detail below.

### SUMMARY OF THE INVENTION

The present invention consists of a method of synthesizing ethers of anhydrosugars. An anhydrosugar is dissolved in a solvent or blend of solvents and an aqueous caustic base is added to the solution. The solvent water is distilled out and the water formed in the reaction by azeotropic distillation with one or more of the solvents to form a slurry of the insoluble alkali metal alkoxide. An alkyl or aralkyl halide, and a sulfonate ester of the equivalent alcohol, are added to the alkoxide in a polar organic solvent, optionally in the presence of a specified crown ether. The mixture is heated to effect the ether-forming reaction and the product is separated from the by-product alkali metal salt. Any remaining solvent is removed and the product is purified as necessary.

The anhydrosugar is preferably a dianhydrosugar, more preferably an isohexitol, and most preferably isosorbide, isomannide, or isoidide.

The solvent is preferably a mixture of one or more polar solvents selected from the group consisting of dimethyl formamide, NN-dimethylacetamide, N-methylpyrrolidone and dimethyl sulfoxide, and one or more hydrocarbons selected from the group consisting of benzene, toluene, xylene, and cumene.

The base is preferably selected from the group consisting of lithium hydroxide, sodium hydroxide, and potassium hydroxide.

The the alkyl halide is preferably n-hexyl bromide, n-dodecyl bromide, 2-ethylhexyl bromide, n-octadecyl bromide, n-hexyl methanesulfonate, n-dodecyl methanesulfonate, 2-ethylhexyl methanesulfonate, n-octadecyl methanesulfonate, n-hexyl toluene-p-sulfonate, n-dodecyl toluene-p-sulfonate, 2-ethylhexyl toluene-p-sulfonate, n-octadecyl toluene-p-sulfonate, benzyl chloride, benzyl bromide, epichlorhydrin or epibromohydrin.

### DETAILED DESCRIPTION OF PREFERRED EMBODIMENTS

The invention consists of novel methods of synthesizing ethers of anhydrosugars, particularly bisanhydrohexitols especially isosorbide, isomannide, and isoidide, each of which may be derived from renewable resources, such as plant-derived glucose, using aqueous base in a mixed solvent system and removing the water by azeotropic distillation

### EXAMPLE 1

### Preparation of Isosorbide Di(n-Hexyl)Ether using Aqueous Alkali

A 3000 ml four-neck flask was fitted with a Dean-Stark tube, reflux condenser with exit bubbler tube, a 250 ml pressure-equalizing dropping funnel with a precision PTFE needle-valve tap, long stem thermometer, gas inlet and sealed paddle stirrer with a PTFE blade. All ground glass joints were protected with PTFE plastic sleeves to prevent the glass-to-glass joints from causing the seizure of such joints due to etching of the borosilicate glass by the strong aqueous caustic alkali. The flask was charged with 73 g (0.50 moles) isosorbide, 500 ml dimethyl acetamide (DMAC) and 500 ml toluene. A slow stream of nitrogen was passed through the apparatus to prevent oxidation. A solution of 40 g sodium hydroxide (1.00 moles) in 40 ml water was placed in the dropping funnel. The system was well sparged with inert gas before any heat was applied, it having been found that isosorbide very easily discolors under alkaline conditions in the presence of oxygen. The mixture was stirred at 200 rpm and brought to a fairly brisk reflux (still head temperature 112° C.). Before any alkali was added, a small amount of water (ca 0.50 ml) collected in the Dean-Stark tube, presumably water of hydration from the isosorbide. The caustic soda solution then added slowly drop-wise to the flask over 2 hours and the mixture stirred briskly and refluxed as water collected in the Dean-Stark tube. The reaction flask (“pot”) temperature was 125° C. and the still head temperature 110° C. As soon as the caustic soda was added, the mixture darkened from pale yellow to deep amber, despite the nitrogen atmosphere. After a few minutes, the reaction mixture suddenly became very foamy and frothy, which is why a large reaction flask was necessary, and there was a mildly exothermic reaction. After thirty minutes, a total of 18 ml water had been collected and the still head had fallen to 101° C. while the pot temperature had fallen to 113° C. Water was steadily removed by azeotropic distillation. About this time, the appearance of the reaction mixture changed sharply and it became opaque and much paler in color. Water continued to evolve for a total of four hours and at the end of this time the mixture had become a thick, cream-colored slurry of the insoluble bis-alkoxide of isosorbide.

By this time the still head had risen to 115° C. and the pot temperature was 125° C. Tiny amounts of water were still being evolved and collecting in the Dean-Stark tube and when the rate had fallen to about 1.8 ml/hr, the reaction was ended. Approximately 56 mls of water had collected (theory 40+18=58 mls). The toluene was distilled out from the reaction flask until 460 mls had been distilled over (b.p. 113° C.) and when the still head rose to 138° C. and the pot temperature had reached 143° C., the reaction was allowed to cool and stand under a slow nitrogen stream overnight.

The next day a test sample taken from the reaction flask was strongly alkaline. A small amount of 15-crown-5 ether (specific for sodium cation) was added (0.5 ml) and then 180 gm (1.09 moles) of n-hexyl bromide added slowly to the cold stirred mixture over 1-2 hours. There was no apparent exotherm, so the mixture was brought to a gentle reflux in DMAC under nitrogen for a total of 5-6 hours, then left to cool to room temperature. At the end of this time the pH of the reaction mixture was 7.0. The precipitated solid (sodium bromide) was filtered off on a sintered glass funnel and washed with acetone. The combined filtrates were taken down on the rotary evaporator to remove solvents, and to remove the DMAC, the final conditions were 60° C. and 17 mBar pressure. The residue was an oily dark amber liquid, which was cloudy with precipitated solid. The product was filtered again through a fine grade glass sinter to remove this solid, leaving a clear oily liquid which weighed 149.0 g (94.9% theoretical). The washed and dried combined yield of solid sodium bromide was 92 g (89%). The di-n-hexyl ether was immiscible with water and formed an upper layer, but on shaking formed a milky emulsion which only slowly separated out again after 1 hour.

### EXAMPLE 2

### Synthesis of Isosorbide 2,5-Dibenzyl Ether using Azeotrope Method to form the Bisalkoxide of Isosorbide

A 1000 ml 4-neck flask was fitted with Dean-Stark head, reflux condenser, paddle stirrer and a 50 ml pressure equalizing tap-funnel. A long-stem thermometer dipping into the liquid was fitted. The system was run under a nitrogen atmosphere. The flask was charged with isosorbide (29.2 g, 0.20 moles), dimethylacetamide (200 ml) and toluene (200 ml). The mixture was heated to reflux and a solution of sodium hydroxide (18.0 g, 0.45 moles) in 18.ml water added dropwise slowly. The reaction mixture went thick and dark and the batch temperature was 115 C. Water distilled out into the Dean-Stark trap. Gradually the mixture in the flask became a cream-colored slurry and no more water distilled out. Excess toluene was allowed to distill until the batch reached 160° C. The final yield of water at this point was 27.0 ml, calculated water produced in the reaction: 27−18=9 ml, theoretical yield of water 7.2 ml.

The batch was cooled to 140° C. and 1.0 ml of 15-crown-5 ether added to the creamy slurry of sodium alkoxide. Benzyl bromide (86 g, 0.50 moles) was added dropwise over 2-3 hours and after addition was complete, the mixture was heated at 140-145° C. for 9-12 hours. It was left to cool overnight. The sodium bromide formed a solid mass on the bottom of the flask with a clear brown supernatant liquid. Dichloromethane was added and the resulting slurry filtered through a fine porosity sintered funnel and the solid on the filter washed with dry dichloromethane. The yield of sodium bromide was 37.5 g, (90% theory). The filtrate was evaporated on the Rotavapor and the last traces of the DMAC removed using a high vacuum (1.0 mbar) and an oil bath at 110° C. until a brown oily residue was left. NMR indicated the presence of isosorbide acetates. The brown oil was stirred with 500 ml of 5% aqueous potassium hydroxide for one hour. Extraction with 3×250 ml of methyl-t-butyl ether yielded 30 g of crude dibenzyl ether, 46% theory.

### EXAMPLE 3

### Synthesis of Isosorbide 2,5-Diallyl Ether using Azeotrope Method to form the Bisalkoxide of Isosorbide

A 500 ml 3-neck flask was fitted with Dean-Stark head, reflux condenser, paddle stirrer and a 125 ml pressure equalizing tap-funnel. The system was run under a nitrogen atmosphere. The flask was charged with isosorbide (14.4 g, 0.10 moles), dimethylacetamide (100 ml) and toluene (100 ml). The mixture was heated to reflux and a solution of potassium hydroxide (14.9 g of 85% KOH, 0.225 moles) in water to make 25 ml added dropwise over 20 minutes. Water distilled out into the Dean-Stark trap. Gradually the mixture in the flask became a cream-colored slurry and no more water distilled out. The final yield of water at this point was 26.0 ml, calculated water produced in the reaction: 26−12=14 ml, theoretical yield of water 3.6 ml. The water was removed from the Dean Stark trap and the trap filled with toluene (ca. 25 ml.)

The batch was maintained at reflux temperature (ca. 120° C. at top of trap) for one hour. Allyl bromide (37.25 g, 0.25 moles) was added dropwise over 20 minutes and the reaction mixture heated at reflux for 10 minutes before cooling to room temperature. Stirring was stopped after 1 hour. The sodium bromide formed a solid mass on the bottom of the flask with a clear brown supernatant liquid. Dichloromethane was added and the resulting slurry filtered through a fine porosity sintered funnel and the solid on the filter washed with dry dichloromethane. The yield of potassium bromide was 25.7 g, (theoretical, 0.2 moles, 25.2 g). The filtrate was evaporated on the Rotavapor until no more solvent distilled at 10 mm Hg and a bath temperature of 85° C. The resulting golden yellow oil weighted 21.15 g. ¹H NMR analysis of the product showed that it contained 4.2 g of product calculated as diallyl isosorbide (20% yield) in addition to residual dimethyl acetamide and isosorbide acetates

The basic process can be used to make a wide variety of ethers of anhydrosugars, for example, isosorbide di(n-dodecyl)ether, isosorbide di(n-octadecyl), isosorbide dibenzyl ether, and isosorbide diglycidyl ether can be formed following a similar procedure using n-dodecyl bromide, n-octadecyl bromide, benzyl bromide and glycidyl bromide, respectively. The formation of an emulsion indicates that the compounds, particularly the mono-substituted compounds, may have use as non-ionic surfactants.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art that have the disclosure before them will be able to make modifications and variations therein without departing from the scope of the invention.

We claim:

1. A method of synthesizing ethers of anhydrosugars, comprising the steps of:
(a) dissolving an anhydrosugar in a solvent or blend of solvents;
(b) adding an aqueous caustic base to the solution;
(c) distilling out the solvent water and the water formed in the reaction by azeotropic distillation with one or more of the solvents to form a slurry of the insoluble alkali metal alkoxide;
(d) adding a compound selected from the group consisting of alkyl or aralkyl halide, and a sulfonate ester of the equivalent alcohol, to the alkoxide in a polar organic solvent, optionally in the presence of a specified crown ether, and heating the mixture to effect the ether-forming reaction; and
(e) separating the product from the by-product alkali metal salt and removing any remaining solvent and purifying the product as necessary.

2. The method of claim 1, wherein the anhydrosugar is a dianhydrosugar.

3. The method of claim 2, wherein the dianhydrosugar is an isohexitol.

4. The method of claim 3, wherein the isohexitol is selected from the group consisting of isosorbide, isomannide, and isoidide.

5. The method of claim 1 wherein the solvent is a mixture of a polar solvent selected from the group consisting of dimethyl formamide, NN-dimethylacetamide, N-methylpyrrolidone and dimethyl sulfoxide, and a hydrocarbon selected from the group consisting of benzene, toluene, xylene, and cumene.

6. The method of claim 1, wherein the base is selected from the group consisting of lithium hydroxide, sodium hydroxide and potassium hydroxide.

7. The method of claim 1, wherein the alkyl halide is selected from the group consisting of n-hexyl bromide, n-dodecyl bromide, 2-ethylhexyl bromide, n-octadecyl bromide, n-hexyl methanesulfonate, n-dodecyl methanesulfonate, 2-ethylhexyl methanesulfonate, n-octadecyl methanesulfonate, n-hexyl toluene-p-sulfonate, n-dodecyl toluene-p-sulfonate, 2-ethylhexyl toluene-p-sulfonate, n-octadecyl toluene-p-sulfonate, benzyl chloride, benzyl bromide, epichlorhydrin and epibromohydrin.
